(19) United States
(12) Patent Application Publication
(10) Pub. No.: US 2026/0014457 A1
(43) Pub. Date: Jan. 15, 2026

(54) ELECTRICAL STIMULATION DEVICE, CONTROL SYSTEM, AND CONTROL METHOD

(71) Applicant: Murata Manufacturing Co., Ltd., Nagaokakyo-shi (JP)

(72) Inventors: Kazuki MATSUI, Nagaokakyo-shi (JP); Jian LUAN, Nagaokakyo-shi (JP); Kazuma AOYAMA, Nagaokakyo-shi (JP)

(73) Assignee: Murata Manufacturing Co., Ltd., Nagaokakyo-shi (JP)

(21) Appl. No.: 19/329,811

(22) Filed: Sep. 16, 2025

Related U.S. Application Data
(63) Continuation of application No. PCT/JP2024/009515, filed on Mar. 12, 2024.

(30) Foreign Application Priority Data
Mar. 17, 2023 (JP) 2023-043359

(51) Int. Cl. A63F 13/28; A61N 1/04; A61N 1/36; A63F 13/98

(52) U.S. Cl. CPC A63F 13/28; A61N 1/0456; A61N 1/36025; A61N 1/36034; A63F 13/98; A63F 2250/497

(57) ABSTRACT

The present disclosure provides an electrical stimulation apparatus, a control system, and a control method that can reduce inflammation caused by the application of a current to a plurality of electrodes adhered to skin. An electrical stimulation apparatus according to the present disclosure is an electrical stimulation apparatus that provides electrical stimulation to a human body. The electrical stimulation apparatus includes a plurality of electrodes to be adhered to skin of a user and configured to provide electrical stimulation, an output circuit configured to apply a current to the plurality of electrodes, and a control circuit configured to control the current to be applied to the plurality of electrodes by the output circuit. The control circuit is configured to apply a current that is less than or equal to a perception threshold of the user to the plurality of electrodes at a predetermined timing.

### CROSS REFERENCE TO RELATED APPLICATIONS

The present application is a continuation of International Application No. PCT/JP2024/009515, filed Mar. 12, 2024, which claims priority to Japanese patent application 2023-043359, filed Mar. 17, 2023, the entire contents of each of which being incorporated herein by reference.

### TECHNICAL FIELD

The present disclosure relates to an electrical stimulation apparatus, a control system, and a control method.

### BACKGROUND ART

In recent years, research and development for evoking an acceleration sensation in a pseudo manner by providing electrical stimulation to a vestibular organ (also simply referred to as the vestibule) of the inner ear have been conducted. In Japanese Unexamined Patent Application Publication No. 2019-017676 (Patent Document 1), motion sickness is suppressed by changing the target provided with electrical stimulation from the vestibular organ that recognizes a movement sensation to the cerebral cortex that integrates vision-based recognition and recognition of the movement sensation by the vestibule.

### CITATION LIST

### Patent Document

Patent Document 1: Japanese Unexamined Patent Application Publication No. 2019-017676

### SUMMARY

### Technical Problems

To provide electrical stimulation to the vestibular organ, the cerebral cortex, or the like, an electrical stimulation apparatus such as the one described in Patent Document 1 applies a current to electrodes adhered to the skin of the head. When the electrical stimulation apparatus continuously provides electrical stimulation to the vestibular organ, the cerebral cortex, or the like for a long time, the charge concentrates between the skin and the electrodes. This may cause skin inflammation. Particularly in relation to a virtual reality (VR) game machine or the like, situations are possible where the electrical stimulation apparatus provides electrical stimulation to the vestibular organ, the cerebral cortex, or the like of a user for a long time. In relation to tDCS (transcranial direct current stimulation), situations are also possible where the electrical stimulation apparatus provides electrical stimulation to the cerebral cortex or the like of a user for a long time.

Accordingly, the present disclosure is directed to providing an electrical stimulation apparatus, a control system, and a control method that can reduce inflammation caused by the application of a current to a plurality of electrodes adhered to the skin.

### Solutions to Problems

An electrical stimulation apparatus according to one embodiment of the present disclosure is an electrical stimulation apparatus that provides electrical stimulation to a human body. The electrical stimulation apparatus includes a plurality of electrodes, an output circuit, and a control circuit. The plurality of electrodes are adhered to skin of a user and provide electrical stimulation. The output circuit applies a current to the plurality of electrodes. The control circuit controls the current to be applied to the plurality of electrodes by the output circuit. The control circuit applies a current that is less than or equal to a perception threshold of the user to the plurality of electrodes at a predetermined timing.

A control system according to one embodiment of the present disclosure is a control system including the electrical stimulation apparatus described above and the external apparatus to be connected to the electrical stimulation apparatus. The electrical stimulation apparatus is an apparatus that provides electrical stimulation to a vestibular organ to evoke an acceleration sensation in the user in a pseudo manner. The external apparatus includes an operation circuit, a display device, and a control device. The operation circuit receives an operation of the user. The display device displays an image to the user. The control device causes the display device to display an image in which an object placed in a virtual space moves in accordance with an operation input value received by the operation circuit. The control device outputs a control signal to the electrical stimulation apparatus. The control signal causes a current for making the user feel a pseudo-acceleration sensation to be applied to the plurality of electrodes, based on a movement of the object in the virtual space.

A control method for an electrical stimulation apparatus according to one embodiment of the present disclosure is a control method for an electrical stimulation apparatus that provides electrical stimulation to a human body. The electrical stimulation apparatus includes a plurality of electrodes, an output circuit, and a control circuit. The plurality of electrodes are adhered to skin of a user and provide electrical stimulation. The output circuit applies a current to the plurality of electrodes. The control circuit controls the current to be applied to the plurality of electrodes by the output circuit. The control method for the electrical stimulation apparatus includes a step of setting a predetermined timing at which a current that is less than or equal to a perception threshold of the user is to be applied to the plurality of electrodes; and a step of applying the current that is less than or equal to the perception threshold of the user to the plurality of electrodes at the set predetermined timing.

### Advantageous Effects

According to the present disclosure, the current that is less than or equal to the perception threshold of the user is applied to the plurality of electrodes at the predetermined timing. This can reduce inflammation caused by the application of the current to the plurality of electrodes adhered to the skin.

### BRIEF DESCRIPTION OF DRAWINGS

FIG. 1 is a schematic diagram of a control system according to an embodiment.

FIG. 2 is a diagram for describing an operation of the control system according to the embodiment.

FIG. 3 is a diagram illustrating a current pattern to be applied to electrodes by a vestibular electrical stimulation apparatus according to the embodiment.

FIG. 4 is a flowchart for describing an operation of the vestibular electrical stimulation apparatus according to the embodiment.

### DESCRIPTION OF EMBODIMENTS

An electrical stimulation apparatus, a control system, and a control method according to an embodiment will be described in detail below with reference to the drawings. In the drawings, the same reference signs denote the same or corresponding parts. An electrical stimulation apparatus described below is, for example, a vestibular electrical stimulation apparatus (GVS: Galvanic Vestibular Stimulation) for evoking an acceleration sensation in a pseudo manner. The electrical stimulation apparatus applies a current to electrodes adhered to the skin surface on the mastoid processes of the human body, and thus can provide electrical stimulation to the vestibular organ. However, the electrical stimulation apparatus is not limited to the vestibular electrical stimulation apparatus. For example, the electrical stimulation apparatus can also be applied to apparatuses such as a transcranial direct current stimulation (tDCS) apparatus that provides electrical stimulation to the head via electrodes adhered to the skin of the head and a low-frequency therapeutic device that provides electrical stimulation to a human body via electrodes adhered to the skin of the human body.

### Embodiment

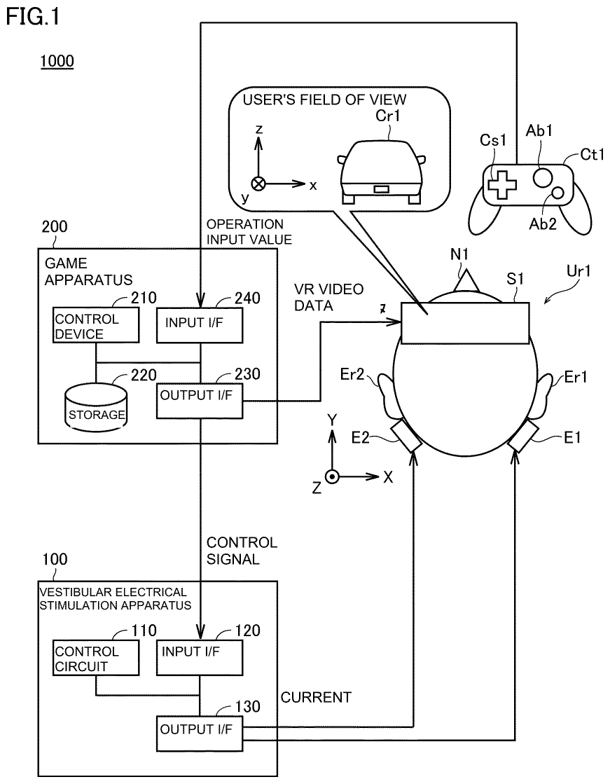

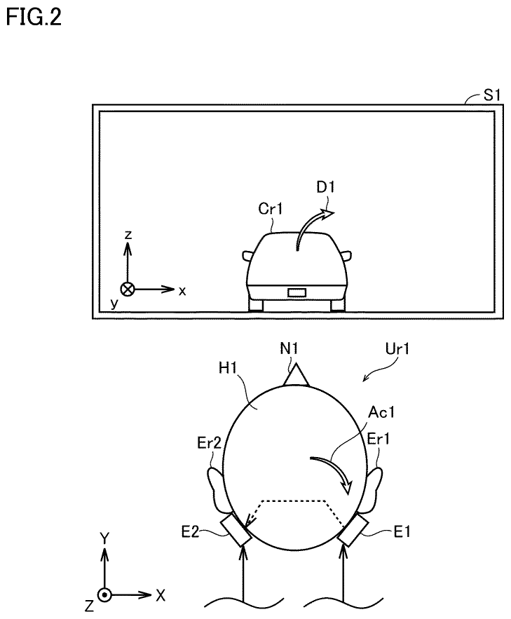

FIG. 1 is a schematic diagram of a control system 1000 according to an embodiment. FIG. 2 is a diagram for describing an operation of the control system 1000 according to the embodiment. The control system 1000 is a game system that uses a vestibular electrical stimulation apparatus 100 and a game apparatus 200 (external apparatus) in combination to make a user Ur1 who plays a game with the game apparatus 200 feel an acceleration sensation in a pseudo manner with the vestibular electrical stimulation apparatus 100. The game apparatus 200 described below is a VR game apparatus that shows a virtual space to the user Ur1 by using a head-mounted display (HMD) and allows the user Ur1 to operate an object (e.g., Cr1) in the virtual space.

By outputting a control signal to the vestibular electrical stimulation apparatus 100, the game apparatus 200 can make, using the vestibular electrical stimulation apparatus 100, the user Ur1 feel pseudo-accelerations produced on an object in accordance with the object that moves in the virtual space. As a result, compared with a simple VR game apparatus, the game apparatus 200 can further increase the user Ur1's sense of immersion into the game by making the user Ur1 feel the pseudo-accelerations produced on the object. Note that the game apparatus 200 is not limited to a VR game apparatus that uses a head-mounted display, and may be a game apparatus that uses a two-dimensional display such as a liquid crystal display or an organic EL display.

The vestibular electrical stimulation apparatus 100 includes electrodes E1 and E2, a control circuit 110, an input interface 120, and an output interface 130. In the vestibular electrical stimulation apparatus 100, the electrodes E1 and E2 are attached to the user Ur1 as illustrated in FIG. 1. Note that FIG. 1 illustrates the user Ur1 viewed from above, and illustrates a head H1, a right ear Er1, a left ear Er2, and a nose N1 of the user Ur1. The electrodes E1 and E2 are also referred to as electrode pads.

FIG. 1 illustrates X, Y, and Z axes representing three-axis directions of a real space where the user Ur1 is present. Among the X, Y, and Z axes, a vertical direction with respect to a floor surface on which the user Ur1 is standing is defined as a “Z-axis direction”, a direction that is perpendicular to the Z-axis direction and to which the user Ur1 faces is defined as a “Y-axis direction”, and a direction that is perpendicular to both the Y-axis direction and the Z-axis direction is defined as an “X-axis direction”. A positive Z-axis direction may be referred to as an upper side. A negative Z-axis direction may be referred to as a lower side. A positive Y-axis direction may be referred to as a front side. A negative Y-axis direction may be referred to as a rear side. A positive X-axis direction may be referred to as a right side. A negative X-axis direction may be referred to as a left side.

The vestibular electrical stimulation apparatus 100 is an apparatus that provides electrical stimulation to the vestibular organ of the user Ur1 to produce a pseudo-acceleration sensation in the user Ur1. The vestibular organ includes hair cells that are in the semicircular canals and in the utricle and saccule of the otolith organ. The semicircular canals receive rotational angular accelerations with respect to the three axes, while the utricle and saccule receive linear accelerations. The vestibular electrical stimulation apparatus 100 applies a current from the output interface 130 to the electrodes E1 and E2 attached to the user Ur1, and thus can provide the user Ur1 with an acceleration sensation corresponding to the current value.

The control circuit 110 is, for example, a hard-wired circuit such as an ASIC (Application Specific Integrated Circuit) or an FPGA (Field-Programmable Gate Array). The control circuit 110 may include a CPU (Central Processing Unit) and a memory such as a RAM (Random Access Memory).

The input interface 120 is connected to the game apparatus 200. Thus, the vestibular electrical stimulation apparatus 100 receives a control signal from the game apparatus 200 via the input interface 120. The control signal includes electrical stimulation data or the like. Based on the input control signal, the control circuit 110 calculates a value of the current to be applied to the electrodes E1 and E2. The output interface 130 is connected to the electrodes E1 and E2 by wires, and applies the current having the value calculated by the control circuit 110 to the electrodes E1 and E2.

The electrode E1 is attached to an area behind the right ear Er1 of the user Ur1, and the electrode E2 is attached to an area behind the left ear Er2 of the user Ur1. More specifically, the electrode E1 is attached to the mastoid process adjacent to the right ear Er1, and the electrode E2 is attached to the mastoid process adjacent to the left ear Er2. The electrodes E1 and E2 have, for example, a hydrogel on the surfaces thereof, and thus can be adhered to the mastoid processes. Obviously, the electrodes E1 and E2 may be pressed by earmuffs or the like so that the electrodes E1 and E2 do not detach from the mastoid processes. The positions where the electrodes E1 and E2 are adhered may be positions that are non-hairy areas free from hair, beard, or the like.

As described above, the electrodes E1 and E2 are respectively arranged at the right and left mastoid processes, so that the vestibular electrical stimulation apparatus 100 can provide the user Ur1 with a pseudo-acceleration sensation in the X-axis direction. The vestibular electrical stimulation apparatus 100 may provide the user Ur1 with pseudo-acceleration sensations in the Y-axis and Z-axis directions or for the roll, pitch, and yaw, by changing the arrangement of the electrodes E1 and E2 and the number of electrodes.

The game apparatus 200 includes a display device S1, a controller Ct1 for games, a control device 210, a storage 220, an input interface 240, and an output interface 230. In the embodiment, the description will be given below on the assumption that a racing game for driving a car is executed on the game apparatus 200.

The display device S1, which is a head-mounted display, displays VR video data such that the rear portion of a car Cr1 is present at the center of the field of view of the user Ur1. Obviously, the display device S1 may display VR video data from the first-person perspective of the user Ur1 who is riding in the car Cr1. As illustrated in FIG. 1, the display device S1 also displays x, y, and z axes. The x, y, and z axes displayed on the display device S1 represent the three-axis directions in the virtual space. The three-axis directions in the virtual space correspond to the three-axis directions in the real space. A positive z-axis direction is referred to as an upper side. A negative z-axis direction is referred to as a lower side. A positive y-axis direction is referred to as a front side. A negative y-axis direction is referred to as a rear side. A positive x-axis direction is referred to as a right side. A negative x-axis direction is referred to as a left side. The car Cr1 is traveling straight in the positive y-axis direction.

The controller Ct1 at least includes a cross control Cs1 and buttons Ab1 and Ab2. The cross control Cs1 is a control for operating the position of the car Cr1 (object) to be operated by the user Ur1. The cross control Cs1 may be a key or button of another form such as a stick-shaped input device, or an input device such as a gesture input device or a steering-wheel-shaped input device. The user Ur1 operates the controller Ct1, and thus can move the car Cr1 displayed in front of them forward or change the traveling direction of the car Cr1.

More specifically, the user Ur1 presses the button Ab1 to move the car Cr1 forward. The user Ur1 presses a right button of the cross control Cs1 to turn the car Cr1 to the right, and presses a left button of the cross control Cs1 to turn the car Cr1 to the left. The left and right buttons of the cross control Cs1 correspond to the steering wheel of a car in the real space. The button Ab1 corresponds to the accelerator of the car in the real space. The button Ab2 corresponds to the brake of the car in the real space. An operation performed by the user Ur1 on the controller Ct1 is input as an operation input value to the control device 210 via the input interface 240. The controller Ct1 and the input interface 240 correspond to an “operation circuit” of the game apparatus 200.

The control device 210 includes, for example, a processor such as a CPU or an MPU that executes a game program, and a memory such as a RAM. The control device 210 may be a hard-wired circuit such as an ASIC or an FPGA.

The storage 220 is implemented by a rewritable nonvolatile memory or magnetic disk such as an HDD (Hard Disk Drive), an SSD (Solid State Drive), or a flash memory. The storage 220 stores a program of the racing game of the car Cr1 illustrated in FIG. 1. Based on the program, the control device 210 causes the display device S1 to display the car Cr1, which is an object. Specifically, the control device 210 sends VR video data to the display device S1 via the output interface 230.

An operation of the control system 1000 will be described next. FIG. 2 is a diagram for describing the operation of the control system 1000 according to the embodiment. Upon receiving an operation of the user Ur1 for pressing the right button of the cross control Cs1, the game apparatus 200 turns the car Cr1 displayed on the display device S1 in a right direction D1 (the positive x-axis direction). The vestibular electrical stimulation apparatus 100 applies a current to the electrodes E1 and E2 to make the user Ur1 feel an acceleration in the right direction in accordance with the display of the car Cr1 turning in the right direction D1.

Specifically, to evoke a pseudo-acceleration sensation in the user Ur1 so that the user Ur1 can feel turning in the right direction, the vestibular electrical stimulation apparatus 100 applies a current such that the electrode E2 serves as a negative terminal and the electrode E1 serves as a positive terminal. When the current flows from the electrode E1 to the electrode E2, the current flows through the inside of the earhole of the user Ur1 and provides electrical stimulation to the vestibular organ. This can cause a sensation of an acceleration Ac1 in the roll direction in the user Ur1. From the viewpoint of safety, the current that flows between the electrode E1 and the electrode E2 may have a value of 4.0 mA or less.

Thus, with the vestibular electrical stimulation apparatus 100, the control system 1000 can make the user Ur1 feel a pseudo-acceleration sensation of turning in the right direction in accordance with the image of the car Cr1, displayed in the game on the game apparatus 200, turning in the right direction. Therefore, compared with simply showing the user Ur1 an image of a car turning in the right direction in a game, showing the user Ur1 the image of the car turning in the right direction with the vestibular electrical stimulation apparatus 100 providing the user Ur1 with a pseudo-acceleration sensation can increase the user Ur1's sense of immersion into the game.

It is considered that when a current for providing electrical stimulation is applied to the electrodes E1 and E2, charge exchange is taking place at an interface between the skin and the electrodes E1 and E2. Therefore, when the user Ur1 plays a game on the game apparatus 200 for a long time, the vestibular electrical stimulation apparatus 100 keeps providing electrical stimulation to the vestibular organ for a long time. Consequently, the charge concentrates between the skin and the electrodes E1 and E2. This may cause skin inflammation. Therefore, to remove the charge accumulated between the skin and the electrodes E1 and E2, the control system 1000 applies a current (e.g., noise current) that is less than or equal to a perception threshold of the user Ur1 to the electrodes E1 and E2 at a predetermined timing.

In particular, when the vestibular electrical stimulation apparatus 100 provides current stimulation, the electrodes E1 and E2 are adhered to the head. Thus, if skin inflammation occurs, the inflammation is more noticeable than that caused when the electrodes E1 and E2 are adhered to other body parts. Therefore, an improvement is desired. Accordingly, to remove the charge accumulated between the skin and the electrodes E1 and E2, the vestibular electrical stimulation apparatus 100 provides a noise current (nGVS: noisy Galvanic Vestibular Stimulation), which is a current that is reciprocally applied between the electrodes E1 and E2 and is less than or equal to the perception threshold of the user Ur1.

Specifically, the current that is less than or equal to the perception threshold of the user Ur1 is a noise current having a current density less than 0.5 A/m². Obviously, depending on the user Ur1, even a noise current having a current density of 0.4 A/m² may be perceived. In this case, a noise current having a lower current density may be set. The noise current may be white noise. Obviously, the noise current is not necessarily white noise but may be a signal that can reciprocally apply a current between the electrodes E1 and E2. The current that is less than or equal to the perception threshold of the user Ur1 is applied to dissipate accumulated charge between the skin and the electrodes E1 and E2 and is hereafter referred to as a “charge-dissipating current” This charge-dissipating current is not necessarily a noise current but may be any current that can remove the charge accumulated between the skin and the electrodes E1 and E2. For example, the current that is less than or equal to the perception threshold may be a current (e.g., current having a current density of −0.3 A/m²) that has a polarity opposite to the polarity of the current (e.g., +3 mA) for providing electrical stimulation to the vestibular organ and that is less than or equal to the perception threshold. The noise current is not limited to a current whose center current value is 0 mA, and may be a current whose center current value has a positive or negative polarity. For example, when electrical stimulation is provided to the vestibular organ as a current of +3 mA, the center current value of the noise current (−0.5 mA to +0.1 mA) may be −0.2 mA and have a negative polarity. By applying the noise current to the electrodes E1 and E2, the vestibular electrical stimulation apparatus 100 can remove the charge accumulated between the skin and the electrodes E1 and E2 by the GVS and can suppress skin inflammation. Since the vestibular electrical stimulation apparatus 100 can suppress skin inflammation, electrical stimulation to the vestibular organ can be performed by GVS for a long time.

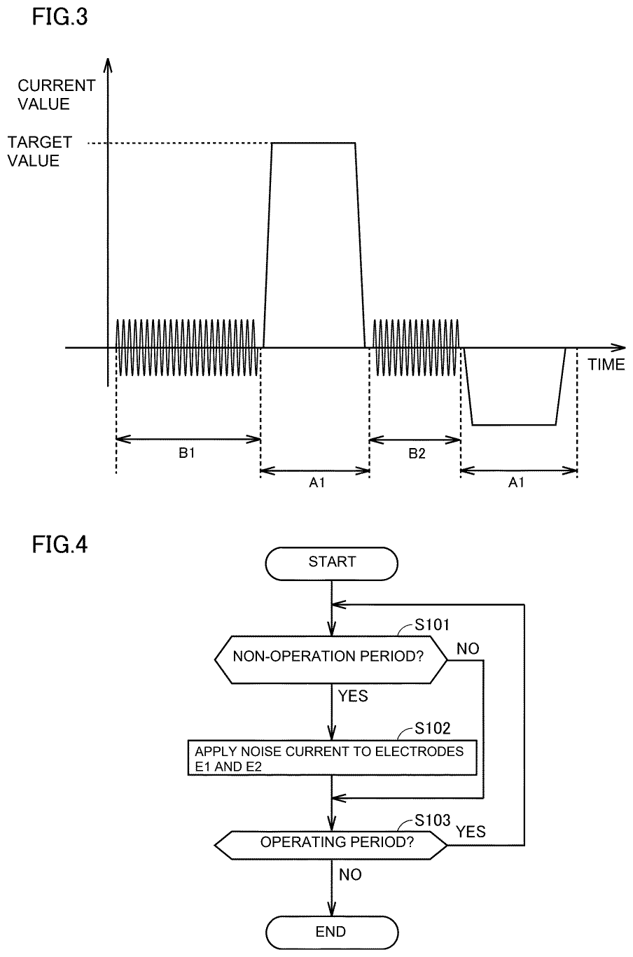

Next, a timing at which the vestibular electrical stimulation apparatus 100 applies the noise current to the electrodes E1 and E2 will be described. To avoid an influence on electrical stimulation provided to the vestibular organ by GVS, the period in which the vestibular electrical stimulation apparatus 100 applies the noise current to the electrodes E1 and E2 may be an operating period of the vestibular electrical stimulation apparatus 100 (e.g., a period from when the game apparatus 200 is turned on to when the game apparatus 200 is turned off) other than a period for which a current for providing electrical stimulation to the vestibular organ is applied to the electrodes E1 and E2. FIG. 3 is a diagram illustrating a current pattern to be applied to the electrodes E1 and E2 by the vestibular electrical stimulation apparatus 100 according to the embodiment. In FIG. 3, the horizontal axis represents the time and the vertical axis represents the current value.

As illustrated in FIG. 3, a period for which the game apparatus 200 is loading the game program (a non-operation period B1 for which the car Cr1 is not operable by the user Ur1) is a period for which the vestibular electrical stimulation apparatus 100 need not provide electrical stimulation to the vestibular organ. Therefore, the vestibular electrical stimulation apparatus 100 applies the noise current to the electrodes E1 and E2. In an operation period A1 in which the game is started on the game apparatus 200 and the car Cr1 is operable by the user Ur1, the vestibular electrical stimulation apparatus 100 needs to provide electrical stimulation to the vestibular organ in accordance with the operation of the car Cr1. Therefore, the vestibular electrical stimulation apparatus 100 does not apply the noise current to the electrodes E1 and E2 to avoid an influence on the electrical stimulation provided to the vestibular organ.

A non-operation period B2 for which the car Cr1 is not operable by the user Ur1 because the game apparatus 200 shows game events or displays a menu is a period for which the vestibular electrical stimulation apparatus 100 need not provide electrical stimulation to the vestibular organ. Therefore, the vestibular electrical stimulation apparatus 100 applies the noise current to the electrodes E1 and E2. In an operation period A2 in which the game is resumed in the game apparatus 200 and the car Cr1 is operable by the user Ur1, the vestibular electrical stimulation apparatus 100 needs to provide electrical stimulation to the vestibular organ in accordance with the operation of the car Cr1. Therefore, the vestibular electrical stimulation apparatus 100 does not apply the noise current to the electrodes E1 and E2 to avoid an influence on the electrical stimulation provided to the vestibular organ.

Note that the vestibular electrical stimulation apparatus 100 may apply the noise current to the electrodes E1 and E2 even in the operation period in which the game is executed on the game apparatus 200 and the car Cr1 is operable by the user Ur1 but the user Ur1 is not operating the car Cr1. When a game is played on the game apparatus 200 continuously for a predetermined time (e.g., one hour), the game may be forcibly interrupted, and the vestibular electrical stimulation apparatus 100 may apply the noise current to the electrodes E1 and E2.

Specifically, the setting of the timing at which the vestibular electrical stimulation apparatus 100 applies the noise current to the electrodes E1 and E2 will be described using a flowchart. FIG. 4 is a flowchart for describing an operation of the vestibular electrical stimulation apparatus 100 according to the embodiment.

First, based on a control signal received from the game apparatus 200 after the game apparatus 200 is turned on, the vestibular electrical stimulation apparatus 100 determines whether a current timing is in the non-operation period in which the car Cr1 is not operable by the user Ur1 on the game apparatus 200 (step S101). The control signal includes information that enables the operation period in which the car Cr1 is operable by the user Ur1 and the non-operation period in which the car Cr1 is not operable by the user Ur1 to be distinguished from each other. When the control signal includes information indicating the operation period, the control signal further includes electrical stimulation data such as a target value, a polarity, and an application period of the current to be applied to the electrodes E1 and E2 in the operation period. The vestibular electrical stimulation apparatus 100 may distinguish between the operation period and the non-operation period, based on whether the control signal includes the electrical stimulation data.

If the current timing is in the non-operation period in which the car Cr1 is not operable by the user Ur1 (YES in step S101), the vestibular electrical stimulation apparatus 100 sets the identified non-operation period as the predetermined timing, and applies the noise current to the electrodes E1 and E2 (step S102). The vestibular electrical stimulation apparatus 100 may continuously apply the noise current to the electrodes E1 and E2 for the non-operation period. Alternatively, the vestibular electrical stimulation apparatus 100 may stop applying the noise current upon an elapse of a certain time (e.g., 60 seconds) even in the non-operation period. The vestibular electrical stimulation apparatus 100 may intermittently apply the noise current to the electrodes E1 and E2 in the non-operation period.

If the current timing is in the operation period in which the car Cr1 is operable by the user Ur1 (NO in step S101), the vestibular electrical stimulation apparatus 100 skips the processing of step S102. The vestibular electrical stimulation apparatus 100 then determines whether the current timing is in the operating period (step S103). The operating period of the vestibular electrical stimulation apparatus 100 is related to the operating period of the game apparatus 200, and is, for example, a period from when the game apparatus 200 is turned on to when the game apparatus 200 is turned off. Obviously, the operating period of the vestibular electrical stimulation apparatus 100 is not necessarily related to the operating period of the game apparatus 200, and may be, for example, a period from when the vestibular electrical stimulation apparatus 100 is turned on to when the vestibular electrical stimulation apparatus 100 is turned off.

If the current timing is in the operating period (YES in step S103), the vestibular electrical stimulation apparatus 100 returns the process to step S101. On the other hand, if the current timing is not in the operating period (NO in step S103), the vestibular electrical stimulation apparatus 100 ends the process of applying the noise current to the electrodes E1 and E2.

### Modification

In the embodiment described above, the control system 1000 has been described as the game system that uses the vestibular electrical stimulation apparatus 100 and the game apparatus 200 (external apparatus) in combination to make the user Ur1 who plays a game with the game apparatus 200 feel an acceleration sensation in a pseudo manner with the vestibular electrical stimulation apparatus 100. However, the control system is not limited to the game system, and may be an amusement system that uses the vestibular electrical stimulation apparatus 100 and a video apparatus (external apparatus) in combination to make the user Ur1 feel an acceleration sensation in a pseudo manner with the vestibular electrical stimulation apparatus 100 in accordance with the movement of an object displayed by the video apparatus.

The vestibular electrical stimulation apparatus 100 may apply a charge-dissipating current to the electrodes E1 and E2 even when the vestibular electrical stimulation apparatus 100 is not connected to an external apparatus but is used alone to alleviate motion sickness.

The electrical stimulation apparatus is not limited to the vestibular electrical stimulation apparatus 100 and may be, for example, an electrical taste apparatus. The electrical taste apparatus is based on, for example, an ion electrophoresis hypothesis, and is an electrical stimulation apparatus that enables control/enhancement of the taste felt by a person by causing taste ions to move in the oral cavity by electrical stimulation. Specifically, the electrical taste apparatus causes a current to flow through an electrode, which serves as a positive terminal and is adhered to the back of the neck, and a straw, which serves as a negative terminal and is inserted into the oral cavity, to control/enhance the taste of substances ingested through the straw. In this electrical taste apparatus, the electrode is also adhered to the back of the neck. Thus, when the electrical taste apparatus is used for a long time, the charge concentrates between the skin and the electrode. This may cause skin inflammation. Therefore, the electrical taste apparatus also applies a current that is less than or equal to the perception threshold (e.g., a noise current) when a current for controlling/enhancing the taste is not being applied. This can reduce inflammation.

### Aspect

(1) An electrical stimulation apparatus according to the present disclosure is an electrical stimulation apparatus that provides electrical stimulation to a human body, the electrical stimulation apparatus including:
a plurality of electrodes to be adhered to skin of a user and configured to provide electrical stimulation;
an output circuit configured to apply a current to the plurality of electrodes; and
a control circuit configured to control the current to be applied to the plurality of electrodes by the output circuit, wherein the control circuit is configured to apply a current that is less than or equal to a perception threshold of the user to the plurality of electrodes at a predetermined timing.
(2) The electrical stimulation apparatus according to (1), wherein the predetermined timing is in an operating period of the electrical stimulation apparatus other than a period for which a current for providing the electrical stimulation is applied to the plurality of electrodes.
(3) The electrical stimulation apparatus according to (1) or (2), wherein the current that is less than or equal to the perception threshold of the user is a current having a current density less than 0.5 A/m².
(4) The electrical stimulation apparatus according to any one of (1) to (3), wherein
the current that is less than or equal to the perception threshold of the user is a current having a current density of an opposite polarity to that of a current for providing the electrical stimulation.
(5) The electrical stimulation apparatus according to any one of (1) to (4), wherein the current that is less than or equal to the perception threshold of the user is a noise current.
(6) The electrical stimulation apparatus according to (5), wherein the noise current is white noise.
(7) The electrical stimulation apparatus according to any one of (1) to (6), further including:
an input circuit configured to receive a control signal from an external apparatus, wherein
the control circuit is configured to set the predetermined timing, based on the control signal received by the input circuit.
(8) A control system according to the present disclosure including:
the electrical stimulation apparatus according to (7); and
the external apparatus to be connected to the electrical stimulation apparatus.
(9) The control system according to (8), wherein
the electrical stimulation apparatus is an apparatus that provides the electrical stimulation to a vestibular organ to evoke an acceleration sensation in the user in a pseudo manner,
the external apparatus includes:
   an operation circuit configured to receive an operation of the user;
   a display device configured to display an image to the user; and
   a control device configured to cause the display device to display an image in which an object placed in a virtual space moves in accordance with an operation input value received by the operation circuit, and
the control device is configured to
   output a control signal to the electrical stimulation apparatus, the control signal causing a current for making the user feel a pseudo-acceleration sensation to be applied to the plurality of electrodes, based on a movement of the object in the virtual space.
(10) The control system according to (9), wherein
the control device is configured to output, to the electrical stimulation apparatus, a control signal for distinguishing between an operation period in which the object is operable by the user and a non-operation period in which the object is not operable by the user, and
the electrical stimulation apparatus is configured to identify the non-operation period, based on the control signal received by the input circuit, and set the identified non-operation period as the predetermined timing.
(11) A control method for an electrical stimulation apparatus according to the present disclosure is a control method for an electrical stimulation apparatus that provides electrical stimulation to a human body,
the electrical stimulation apparatus including: a plurality of electrodes to be adhered to skin of a user and configured to provide electrical stimulation; an output circuit configured to apply a current to the plurality of electrodes; and a control circuit configured to control the current to be applied to the plurality of electrodes by the output circuit, the control method including:
a step of setting a predetermined timing at which a current that is less than or equal to a perception threshold of the user is to be applied to the plurality of electrodes; and
a step of applying the current that is less than or equal to the perception threshold of the user to the plurality of electrodes at the set predetermined timing.

The embodiment disclosed herein should be construed as being illustrative and non-restrictive in every respect. The scope of the present disclosure is defined by the claims, rather than the description above, and is intended to include every modification within the meaning and scope equivalent to the claims.

### REFERENCE SIGNS LIST

100 vestibular electrical stimulation apparatus
110 control circuit
120, 240 input interface
130, 230 output interface
200 game apparatus
210 control device
220 storage
1000 control system

1. An electrical stimulation apparatus that provides electrical stimulation to a human body, comprising:
a plurality of electrodes to be adhered to skin of a user and configured to provide electrical stimulation;
an output circuit configured to apply a current to the plurality of electrodes; and
a control circuit configured to control the current to be applied to the plurality of electrodes by the output circuit, wherein
the control circuit is configured to apply a current that is less than or equal to a perception threshold of the user to the plurality of electrodes at a predetermined timing.

2. The electrical stimulation apparatus according to claim 1, wherein the predetermined timing is in an operating period of the electrical stimulation apparatus other than a period for which a current for providing the electrical stimulation is applied to the plurality of electrodes.

3. The electrical stimulation apparatus according to claim 2, wherein the control circuit is configured to apply the current that is less than or equal to the perception threshold of the user continuously throughout the predetermined timing.

4. The electrical stimulation apparatus according to claim 2, wherein the control circuit is configured to apply the current that is less than or equal to the perception threshold of the user intermittently throughout the predetermined timing.

5. The electrical stimulation apparatus according to claim 1, wherein the current that is less than or equal to the perception threshold of the user is a current having a current density less than 0.5 A/m².

6. The electrical stimulation apparatus according to claim 1, wherein the current that is less than or equal to the perception threshold of the user is a current having a current density of a polarity opposite to that of a current for providing the electrical stimulation.

7. The electrical stimulation apparatus according to claim 1, wherein the current that is less than or equal to the perception threshold of the user is a noise current.

8. The electrical stimulation apparatus according to claim 7, wherein the noise current is white noise.

9. The electrical stimulation apparatus according to claim 1, further comprising:
an input circuit configured to receive a control signal from an external apparatus, wherein
the control circuit is configured to set the predetermined timing, based on the control signal received by the input circuit.

10. A control system comprising:
the electrical stimulation apparatus according to claim 7; and
the external apparatus to be connected to the electrical stimulation apparatus.

11. The control system according to claim 10, wherein
the electrical stimulation apparatus is an apparatus that provides the electrical stimulation to a vestibular organ to evoke an acceleration sensation in the user in a pseudo manner,
the external apparatus includes:
an operation circuit configured to receive an operation of the user;
a display device configured to display an image to the user; and
a control device configured to cause the display device to display an image in which an object placed in a virtual space moves in accordance with an operation input value received by the operation circuit, and
the control device is configured to
output a control signal to the electrical stimulation apparatus, the control signal causing a current for making the user feel a pseudo-acceleration sensation to be applied to the plurality of electrodes, based on a movement of the object in the virtual space.

12. The control system according to claim 11, wherein
the control device is configured to output, to the electrical stimulation apparatus, a control signal for distinguishing between an operation period in which the object is operable by the user and a non-operation period in which the object is not operable by the user, and
the electrical stimulation apparatus is configured to identify the non-operation period, based on the control signal received by the input circuit, and set the identified non-operation period as the predetermined timing.

13. The control system according to claim 12, wherein the non-operation period includes at least one of a game program loading period or a menu display period.

14. The control system according to claim 11, wherein the display device is a head-mounted display.

15. The electrical stimulation apparatus according to claim 1, wherein the electrical stimulation provides Galvanic Vestibular Stimulation (GVS) to evoke a pseudo-acceleration sensation in the user.

16. The electrical stimulation apparatus according to claim 15, wherein the plurality of electrodes are configured to be adhered to mastoid processes of the user.

17. The electrical stimulation apparatus according to claim 1,
wherein the electrical stimulation provides transcranial direct current stimulation (tDCS).

18. A control method for an electrical stimulation apparatus that provides electrical stimulation to a human body,
the electrical stimulation apparatus including: a plurality of electrodes to be adhered to skin of a user and configured to provide electrical stimulation; an output circuit configured to apply a current to the plurality of electrodes; and a control circuit configured to control the current to be applied to the plurality of electrodes by the output circuit, the control method comprising:
setting a predetermined timing at which a current that is less than or equal to a perception threshold of the user is to be applied to the plurality of electrodes;
adhering the plurality of electrodes to skin of the user and
applying the current that is less than or equal to the perception threshold of the user to the plurality of electrodes at the set predetermined timing.

19. The control method according to claim 18, wherein applying includes applying a noise current to the plurality of electrodes.

20. The control method according to claim 18, wherein the predetermined timing is a non-operation period during which a primary electrical stimulation is not applied, the method further comprising: receiving a control signal from an external apparatus indicating the non-operation period.
